(19) United States
(12) Patent Application Publication
(10) Pub. No.: US 2011/0129834 A1
(43) Pub. Date: Jun. 2, 2011

(54) SELECTIVE AMPLIFICATION OF POLYNUCLEOTIDE SEQUENCES

(75) Inventors: Zhoutao CHEN, Carlsbad, CA (US); Hua Zhang, Carlsbad, CA (US)

(73) Assignee: LIFE TECHNOLOGIES CORPORATION, Carlsbad, CA (US)

(21) Appl. No.: 12/953,359

(22) Filed: Nov. 23, 2010

Related U.S. Application Data
(60) Provisional application No. 61/263,988, filed on Nov. 24, 2009.

(51) Int. Cl. C12Q 1/68; C12P 19/34; C40B 40/06

(52) U.S. Cl. 435/6.11; 435/91.2; 506/16

(57) ABSTRACT

The application relates to compositions and methods which may be used for amplifying selected portions of nucleic acid samples. Samples may comprise an entire genome of a bacterium, plant, animal, or other organism. In some instances, methods allow for enrichment of hundred or thousands of target nucleic acids of interest in an efficient and cost effective manner.

### CROSS-REFERENCE TO RELATED APPLICATIONS

This application claims the benefit of U.S. Provisional Application No. 61/263,988, filed Nov. 24, 2009, the contents of which are incorporated herein by reference in its entirety.

### BACKGROUND

Next-generation sequencing technologies have significantly increased the throughput and decreased the cost of large scale sequencing projects such as the sequencing of an entire genome. Despite these advances, the resources needed to sequence a complex mammalian genome remain high and out of the reach of many researchers. Various approaches have been developed to reduce the size and complexity involved in sequencing a genome. These approaches have been based on sequencing only a portion of interest of a genome. Methods for selecting portions of a genome to sequence have included selective amplification of targets of interest and selective capture of targets of interest using either solid phase microarray or solution based hybridization methods.

While existing methods of selective target amplification have been successful in reducing the complexity of samples for sequencing analysis there are difficulties relating to complex sample preparation, cost of reagents and sample size. Many multiplex amplification based enrichment methods make use of complex probes of up to 300 bases which require extensive synthetic procedures to prepare. Other approaches require circular nucleic acid molecules or solid phase based hybridization which may limit efficiency.

### SUMMARY

The invention relates to compositions and methods which may be used, for example, for amplifying selected portions of nucleic acid samples such as samples with comprise the entire genome of an organism (e.g., bacterium, plant, animal, etc.). In some instances, methods described herein allow for the enrichment of hundred or thousands of target nucleic acids of interest. Further, in some instances, this may be done in an efficient and cost effective manner.

Methods described herein may be coupled with high throughput sequencing technologies to allow for the sequencing of nucleic acid regions of interest from large, complex genomes such as mammalian or human genomes. In some instances, such methods may make use of relatively simple, short oligonucleotides (e.g., probes). In many instances, such oligonucleotides can be used in solution based hybridization reactions, leading to cost effective, efficient amplification of desired target sequences.

In some instances, the invention is directed to methods for enrichment of target nucleic acid segments present in samples.

Some embodiments of the invention are directed to methods for amplification of one or more (e.g., one, two, three, five, ten, fifteen, twenty, fifty, one hundred, etc.) target nucleic acid segments and/or molecules. In some instances, the invention includes methods for amplification of one or more target nucleic acid molecule comprising one or more of the following steps: (a) hybridizing the one or more target nucleic acid molecule with a first probe comprising, (i) at the 3′ end, a first target nucleic acid specific sequence, (ii) a first primer sequence at the 5′ end of the first probe and (iii) a tag attached to the 5′ end of the first probe or any part of first primer sequence; (b) extending the first probe; (c) contacting the extended first probe with a solid support comprising a capture molecule which binds the tag; (d) hybridizing the extended first probe with a second probe comprising (i) at the 3′ end, a second a target nucleic acid specific sequence, and (ii) a second primer sequence at a 5′ end of the second probe; (e) extending the second probe; and (f) releasing the extended second probe.

In some embodiments, the extended second probe may be sequenced directly.

In additional embodiments, the extended second probe may be amplified by polymerase chain reaction or other methods known in the art using the first and second primer segments. In further embodiments, the first and second primer segments used for amplification may further comprise sequences used in next generation sequencing applications. For example the first and second primer segments may comprise the P1 and P2 sequences used in SOLiD Sequencing methods, or adaptor sequences used in Genome Sequencer (Roche/454) or Genetic Analyzer (Illumina).

In some embodiments, the amplified extended second probe may be fragmented further and then be used to generate next generation sequencing library by addition of required adaptor sequences for sequence identification. In further embodiments, a signal sequence or barcode which may be a unique nucleotide segment 4-36 bp in length is used to identify a particular nucleotide molecule. This signal sequence may be introduced as a part of adaptor sequence to a sample during next generation sequencing library preparation.

In other embodiments, the extended first probe bound to the solid support may be reused to generate additional copies of the second probe. When additional copies of the second probe are generated, the same or different one or more second probe segments may be used.

Target nucleic acid segments or molecules may be obtained from prokaryotic sources such as bacteria or from eukaryotic sources such as viruses, plants, yeast, molds, invertebrates, insects, vertebrates, fish, mammals, rodents and primates. Nucleic acid may be RNA or DNA and may be single or double-stranded. For embodiments where the nucleic acid is double-stranded the nucleic acid may be denatured before, or as part of, hybridization. In some embodiments, large nucleic acid may be used directly without fragmentation, such as high molecular weight genomic DNA. In additional embodiments, the nucleic acid may be fragmented. Methods of fragmentation may be mechanical such as shearing or sonication or may be enzyme based employing enzymes such as restriction enzymes. The nucleic acid fragments may be from 100 bp to 20,000 bp, 200 bp to 20,000 bp, 300 bp to 20,000 bp, 500 bp to 20,000 bp, 700 bp to 20,000 bp, 1,000 bp to 20,000 bp, 5,000 bp to 20,000 bp, 10,000 bp to 20,000 bp, 15,000 bp to 20,000 bp, 100 bp to 15,000 bp, 100 bp to 10,000 bp, 100 bp to 5,000 bp, 100 bp to 3,000 bp, 100 bp to 1,000 bp, 100 bp to 500 bp, 500 bp to 15,000 bp, 500 bp to 10,000 bp, 500 bp to 5,000 bp, 500 bp to 3,000 bp, etc. in length.

Target specific sequences in the probes may be chosen to hybridize with nucleic acid segments in or near regions of a genome that are of interest (e.g., target nucleic acid segments). As used herein, the term “near” may mean within 10 bp, 50 bp, 100 bp, 500 bp, 1,000 bp, or 2,000 bp of a region of interest in a genome. In further embodiments, target specific sequences in the probes may hybridize with nucleic acid segments that flank a region of interest. As used herein, the term “flank”, when used in reference to probes, may mean that the target specific sequences in the probes which hybridize at or close to (e.g., within 5 bp, 10 bp, 15 bp, 20 bp or 30 bp) one or both ends of a region of interest.

Genomic regions of interest may also encode functional RNA molecules such as microRNAs. Genomic regions of interest may also encode proteins involved in particular metabolic or regulatory pathways such as kinases, proteins or associated with disease states such as oncogenes. Genomic regions of interest may further be regions involved in the regulation of gene expression, such as promoter regions or other nucleic acid segments which bind regulatory molecules. Such regions may be contiguous regions including exons, introns and regulatory regions of genes of interest. Such regions may be multiple non-contiguous regions, such as exons of genes of interest.

In some embodiments, target specific sequences in the probes used in methods of the invention may be 15-150 nucleotides in length. In specific embodiments, such sequences may be 10-150, 20-150, 60-150, 90-150, 40-120, 20-90, 20-80, 20-70, 20-60, 20-50, 25-50, 30-50, 35-50, 20-45, 20-40, 30-40, etc. nucleotides in length. Primer binding sites within probes may be 10-40 nucleotides in length. Thus, primer sequences (e.g., common primer binding sites) may be chosen so that they do not substantially hybridize with regions of interest or with nucleic acid present in genomes of interest. Further, primers (e.g., primers which bind to common primer binding sites) may be 10-40, 15-35, 15-30, 15-25, etc. nucleotides in length.

In some embodiments, a signal sequence which may have a unique nucleotide sequence that is 4-36 bp in length and can be used to identify a particular nucleotide molecule. A signal sequence may be embedded in the first probe between first target specific sequence and first primer segment and/or in the second probe between second target specific sequence and second primer segment.

In other embodiments, the invention includes methods for replication of one or more target nucleic acid segments comprising one or more of the following steps: (a) hybridizing the one or more target nucleic acid molecule with one or more probe, wherein the one or more probe comprises: (i) a target nucleic acid specific sequence, (ii) a tag attached to a 5′ end of the one or more probes, and/or (iii) a signal sequence between the target specific polynucleotide sequence and the tag attached to the 5′ end of the one or more probes; (b) extending the one or more probes enzymatically; and (c) contacting the one or more extended probe with a solid support comprising a capture molecule which bind the tag.

In some embodiments, target nucleic acid segments may be obtained from prokaryotic sources such as bacteria or from eukaryotic sources such as plants, yeast, molds, invertebrates, insects, vertebrates, fish, mammals, rodents and primates. Nucleic acid may be RNA or DNA and may be single or double-stranded. Furthermore, nucleic acid may be pre-amplified or pre-enriched or pre-modified, such as incorporating adaptors. For embodiments where the nucleic acid is double-stranded the nucleic acid may be denatured before, or as part of, hybridization. In some embodiments, the nucleic acid may be fragmented. Methods of fragmentation may be mechanical such as shearing or sonication or other methods commonly known in the art or may be enzyme based such as the use of restriction enzymes or DNase. The nucleic acid fragments may be from 100 bp to 20,000 bp, 200 bp to 20,000 bp, 300 bp to 20,000 bp, 500 bp to 20,000 bp, 700 bp to 20,000 bp, 1,000 bp to 20,000 bp, 5,000 bp to 20,000 bp, 10,000 bp to 20,000 bp, 15,000 bp to 20,000 bp, 100 bp to 15,000 bp, 100 bp to 10,000 bp, 100 bp to 5,000 bp, 100 bp to 3,000 bp, 100 bp to 1,000 bp, 100 bp to 500 bp, 100 bp to 300 bp, 200 bp to 5,000 bp, 200 bp to 1,000 bp, 200 bp to 800 bp, 200 bp to 600 bp, 500 bp to 15,000 bp, 500 bp to 10,000 bp, 500 bp to 5,000 bp, 500 bp to 3,000 bp or 500 bp to 2,000 bp in length.

In some embodiments, immobilized one or more extended probe may be fragmented mechanically or enzymatically to release from the solid support and the released probe may be identified.

In some embodiments, immobilized one or more extended probe may be denatured to release captured single-stranded target nucleic acid from a solid support. In some instances, released nucleic acid segments may be identified.

In some embodiments a signal sequence, which may be a unique nucleotide segment 4-36 bp in length used to identify a particular nucleotide molecule, may place between the probe segment and the ligand.

The invention further includes methods for selectively increasing the abundance of one or more target nucleic acid molecule in a mixture. By “increasing the abundance” is meant that the relative amount of one molecule or set of molecules is over the amount of another molecule or set of molecules. For example, if prior to the practice of methods of the invention 0.1 mM of a first set of nucleic acid molecules is present and 1.0 mM of a second set of nucleic acid molecules is present and after the practice of methods of the invention 0.2 mM of a first set of nucleic acid molecules is present and 0.2 mM of a second set of nucleic acid molecules is present, then the abundance of the first set of nucleic acid molecules may be set to have increased in abundance by 10 fold (the different of the ratios between starting at 0.1 mM versus 1.0 mM (a 1:10 ratio) to 0.2 mM versus 0.2 mM (a 1:1)). Methods of the invention may be used to increased the abundance of desired nucleic acid molecules (e.g., target nucleic acid molecules) from about 5 fold to about 15,000 fold (e.g., from about 5 fold to about 8,000 fold, from about 100 fold to about 8,000 fold, from about 1,000 fold to about 8,000 fold, from about 2,000 fold to about 8,000 fold, from about 3,000 fold to about 8,000 fold, from about 1,000 fold to about 6,000 fold, from about 1,000 fold to about 5,000 fold, from about 1,000 fold to about 4,000 fold, from about 2,000 fold to about 5,000 fold, etc.).

In some aspect, methods of the invention will comprise one or more of the following steps: (a) obtaining a sample which contains one or more (e.g., from 1 to 5,000, from 5 to 5,000, from 50 to 5,000, from 1 to 80, from 2 to 80, from 3 to 80, from 10 to 80, from 10 to 100, from 10 to 500, from 20 to 100, etc.) target nucleic acid molecule and one or more non-target nucleic acid molecule; (b) incubating the sample under conditions suitable for converting double-stranded nucleic acid molecules to single-stranded nucleic acid molecules (e.g., placing the sample in a boiling water bath), thereby forming a first reaction mixture; (c) contacting the first reaction mixture of step (b) with a probe (e.g., a probe having the format of probe A) under conditions suitable to allow for the probe to hybridize to the one or more target nucleic acid molecule (e.g., low, moderate or high stringency conditions), wherein the probe comprises at least a sequence (e.g., a sequence from 10 to 100, from 10 to 80, from 10 to 70, from 10 to 60, from 10 to 50, from 10 to 40, from 15 to 75, etc. nucleotides in length) complementary to the one or more target nucleic acid molecule, a primer binding site, and a tag, thereby forming a second reaction mixture; (d) contacting the second reaction mixture of step (c) with a polymerase under conditions suitable for primer extension to form one or more tagged double-stranded target nucleic acid molecule, wherein the one or more double-stranded target nucleic acid molecule comprise a primer binding site and a tag (e.g., the primer binding site and tag become incorporated into the double-stranded target nucleic acid molecule); (e) contacting the one or more tagged double-stranded target nucleic acid molecule formed in step (d) with a support (e.g., a solid support) under conditions which allow for binding of the one or more double-stranded target nucleic acid molecule to the support; and (f) washing of the support formed in step (e) to remove the one or more non-target nucleic acid molecule.

In particular embodiments, methods of the invention further comprise removal of tagged double-stranded target nucleic acid molecules from supports (e.g., solid supports). Tagged double-stranded target nucleic acid molecules removal from supports may be accomplished by any number of methods, including by use of restriction endonucleases or by alteration of ionic strength.

In additional particular embodiments, tagged double-stranded target nucleic acid molecules may be amplified by polymerase chain reaction before addition to or before (e.g., while attached) or after removal from supports.

Further, any number of methods may be used to prepare nucleic acid molecules prior to entry into methods of the invention (e.g., any of the various steps of methods of the invention). As an example, nucleic acid molecules present in the sample may be fragmented. Fragmentation may occur by methods such as mechanical shearing and limited digestion with a “frequent cutting” restriction endonuclease (e.g., Sau3AI). In many instances, fragmentation of nucleic acid molecules in a sample will be followed by size separation (e.g., by gel electrophoresis, by column chromatography, etc.) to obtain a population of nucleic acid molecules in a desired size range.

In many instances, tags will be used to connect target nucleic acid molecules (e.g., directly or indirectly) to a support (e.g., a solid support). In some instance, the tag may be either biotin or a chemical moiety which contains a reactive azide group.

The invention also provides methods for selective enrichment of groups of nucleic acid molecules present in individual samples. Thus, the invention also provides methods in which reaction mixtures are formed (e.g., the first reaction mixture) is contacted with a mixture of probes which differ in nucleotide sequence. In many instances, such probes will often differ in sequence in the region which is complementary to the one or more target nucleic acid molecule, as well as possibly other regions of the probe (e.g., when a bar code is present). In some instances, sequence differences between probes will be a reflection of sequence differences in target nucleic acid molecules. Thus, probe populations or collection may contain between 5 and 100 (e.g., 5 to 90, 10 to 90, 20 to 90, 30 to 90, 50 to 90, 5 to 60, to 40, etc.) probes which differ in nucleotide sequence (e.g., which differ by at least one nucleotide (e.g., from 1 to 20, 1 to 10, 1 to 5, 2 to 20, 2 to 5, 5 to 10, 5 to 20, etc. nucleotides)).

Set or collections of probes which may be used in the practice of the invention may vary greatly but will typically be structured to function in methods of the invention. Thus, the invention includes collections of probes (e.g., two or more probes, such as from 2 to 100, 2 to 50, 2 to 40, 5 to 100, 10 to 100, 20 to 100, etc.) which differ in nucleotide sequence. In many instances, each probe of such collections will comprise one or more of the following: (a) a sequence complementary to at least part of a naturally occurring nucleic acid molecule, (b) a primer binding site, and (c) a tag. The invention includes such probes, as well as methods for the use of such probes.

While probes described herein may have any number of features, typically a region of sequence complementary to at least part of a naturally occurring nucleic acid molecule will be present. In many instances, this region of sequence complementarity will be of sufficient sequence identity and length to allow for hybridization to either a target nucleic acid molecule and/or a naturally occurring nucleic acid (e.g., one normally found in cells or viruses) to allow for hybridization (e.g., hybridization under conditions described here). In many instances, this region of sequence complementary will be between 10 and 100, 10 and 80, 10 and 50, 10 and 400, 10 and 30, 10 and 20, 6 and 20, 6 and 7, 6 and 8, 8 and 20, 5 and 6, 6 and 30, etc. nucleotides in length. In some instances, this region of sequence complementary may be 6, 7 or 8 nucleotides in length. Further, one or more (e.g., 1, 2, 3, 4, 5, 6, 1-10, 2-10, 3-10, 2-5, 2-4, etc.) mismatches may be present in the region of sequence complementary.

As noted above, probes of the invention may contain a primer binding site. Such primer binding sites may be between 5 and 100 (e.g., 5-8, 6-8, 6-10, 6-12, 10-20, etc.) nucleotides in length. In some instances, primer binding sites may be 6, 7 or 8 nucleotides in length.

Individual probes (e.g., probes which differ in sequence by at least one nucleotide) of probe sets of the invention may be contained in the same container or each contained in different containers.

Further, probes of the invention may be in dry form or in solution (e.g., an aqueous solution).

Additionally, individual probes may contain bar code sequences. Thus, the invention includes sets of probes in which members of the sets contain the same bar codes or different bar codes. Such bar codes may be used to deconvolute sample data when samples are mixed together and used in methods of the invention. Thus, the invention provides probe sets where each member of the set has a different bar code (e.g., for identification of the specific target nucleic acid molecule that the probe bound to) or where each member of the set has the same bar code.

### BRIEF DESCRIPTION OF THE DRAWINGS

FIG. 1: A flow chart depicting one embodiment of a method for the selective amplification of a genomic nucleic acid sample.

FIG. 2: A flow chart depicting one embodiment of a method for the selective replication of a genomic nucleic acid sample.

FIG. 3: A diagram depicting the location of probe sequences used in the selective amplification of the HLA locus. A is the first primer sequence; B is the second primer sequence.

FIG. 4: Depicts some exemplary nucleic acids which may be used in the invention and how they may be prepared. Regions of the nucleic acid molecules associated with the various labels are designated by lines of different thicknesses.

FIG. 5: A chart depicting the enrichment of HLA and/or kinase sequences when using a hybridization temperature of 65° C.

FIG. 6: A chart depicting the enrichment targeted vs. non-targeted regions of HLA and/or kinase sequences when using a hybridization temperature of 65° C.

FIG. 7: A chart depicting the Bioanalyzer electropherogram analysis of product yield when using an improved amplification and cleanup protocol. Arrows show the locations of expected amplicons. A, an electropherogram of multiplex amplification products after 25 cycles of PCR; B, product in A after PureLink PCR micro or AMPure beads cleanup; C, cleaned-up multiplex amplification products of the same targets using improved procedure.

FIG. 8: A chart depicting the Bioanalyzer analysis of product yield for nucleic acid targets of 5 kb or 2 kb in size.

FIG. 9: A chart depicting the Bioanalyzer analysis of product yield using a conventional amplification protocol compared to an embodiment of a selective amplification protocol.

### DETAILED DESCRIPTION

In the description that follows, a number of terms related to nucleic acid technology are used. In order to provide a clear and consistent understanding of the specification and claims the following definitions are provided.

Nucleic Acid Segment: As used herein, the term “nucleic acid segment” refers to a region of a nucleic acid molecule and, in some instances, the nucleic acid molecule itself. For example, a region of a nucleic acid molecule may contain an open reading frame. In an appropriate context, this open reading frame could be referred to as a nucleic acid segment. In instances were a nucleic acid molecule consists only of the open reading frame, this nucleic acid molecule can be referred to as a nucleic acid segment.

Target Nucleic Acid Segment: As used herein, the term “target nucleic acid segment” refers to a nucleic acid segment (e.g., a nucleic acid segment in a sample) which is capable of hybridizing to one or more oligonucleotides (e.g., probes) employed in methods describe herein. Target nucleic acid segment may be DNA (e.g., genomic DNA, cDNA, etc.) or RNA (e.g., viral RNANA, mRNA, tRNA, etc.) and in unaltered form, as present in a sample, or may be fragmented.

Nucleic Acid Molecule: As used herein, the phrase “nucleic acid molecule” refers to a sequence of contiguous nucleotides (riboNTPs, dNTPs, ddNTPs, or combinations thereof) of any length (e.g., complete chromosomes and/or genomes). A nucleic acid molecule may encode a full-length polypeptide or a fragment of any length thereof, or may be non-coding.

Sample: As used herein, the phrase “sample” refers to a composition from which nucleic acid is sought. In some instances, samples used in methods described herein may not contain either nucleic acid or any target nucleic acid segments. When one or more target nucleic acid segments are present, methods described herein will often result in the detection and/or amplification of one or more of these target nucleic acid segments. Exemplary samples include materials derived from plants, animals and microbial cultures.

Oligonucleotide: As used herein, the term “oligonucleotide” refers to a synthetic or natural molecule comprising a covalently linked sequence of nucleotides that are joined by a phosphodiester bond between the 3′ position of the pentose of one nucleotide and the 5′ position of the pentose of the adjacent nucleotide. Oligonucleotide used in methods of the invention will often be associated with (e.g., covalently bound to) a tag, referred to herein as “tagged ologonucleotides.” In many instances, probes will be oligonucleotides.

Tag: As used herein, the term “tag” (e.g., a member of an affinity pair) refers to a chemical group which is capable of engaging in ligand-ligand interactions. Exemplary tags include (1) biotin and (2) antigen and/or antibody for such antigen. Other examples of tags are described elsewhere herein.

Nucleotide Sequence Tag: As used herein, the term “nucleotide sequence tag” refers to a tag which is composed of a nucleotide sequence. Nucleotide sequence tags may be used to capture nucleic acid molecules, for example as described herein.

Polypeptide: As used herein, the term “polypeptide” refers to a sequence of contiguous amino acids of any length. The terms “peptide,” oligopeptide,” or “protein” may be used interchangeably with the term “polypeptide.”

Genome: As used herein, the term “genome” refers to the entire genetic complement of an organism. In the case of eukaryotic organisms, genome refers to the nucleic acid molecules found in both the nucleus of the cell and in the mitichondria. A genome includes both coding and non-coding nucleic acid sequences. Genomes, when appropriate, are composed of both chromosomal and non-chromosomal nucleic acids.

Genomic Nucleic Acid: As used herein, the term “genomic nucleic acid”, when used in reference to eukaryotic cells, refers to chromosomal nucleic acid (e.g., DNA) and other DNA molecules normally present in the nucleus but excludes nucleic acid of parasites such as viruses. When used in reference to prokaryotic cells, genomic nucleic acid refers to chromosomal nucleic acid. When used in reference to viruses, genomic nucleic acid refers to the nucleic acid which makes up the viral genome.

Hybridization: As used herein, the term “hybridization” and “hybridizing,” refer to base pairing of two complimentary single-stranded nucleic acid molecules (RNA and/or DNA) to generate a double-stranded molecule. As used herein, two nucleic acid molecules may hybridize, although base pairing is not completely complementary. Accordingly, mismatched bases do not prevent hybridization of two nucleic acid molecules provided that appropriate conditions, well known in the art, are used.

Probe: As used herein, the term “probe” refers to a nucleic acid which hybridizes to a desired nucleic acid sequence in a genome.

Bar Code: As used herein, the term “bar code” refers to a nucleotide sequence (e.g., a unique nucleotide sequence) that allows a nucleic acid molecule containing this sequence to be identified. In certain aspects, bar codes may be located at a specific position on a larger polynucleotide sequence (e.g., a polynucleotide covalently attached to a bead). In certain embodiments, bar codes can each have a length within a range of from 4 to 36 nucleotides, or from 6 to 30 nucleotides, or from 8 to 20 nucleotides. Bar codes may be designed to have melting temperatures of bar codes which are within 10° C. of one another, within 5° C. of one another, or within 2° C. of one another. Bar codes may also be designed to be members of minimally cross-hybridizing sets. In other words, the nucleotide sequence of each member of such a set is sufficiently different from that of every other member of the set that no member can form a stable duplex with the complement of any other member under stringent hybridization conditions. In many instances, nucleotide sequences of each member of a minimally cross-hybridizing set may differs from those of every other member by at least two nucleotides. Bar code technologies are known in the art and are described in Winzeler et al. Science 285:901 (1999); Brenner, Genome Biol. 1:1 (2000); Kumar et al., Nature Rev. 2:302 (2001); Giaever et al., Proc. Natl. Acad. Sci. USA 101:793 (2004); Eason et al., Proc. Natl. Acad. Sci. USA 101:11046 (2004); and Brenner, Genome Biol. 5:240 (2004).

Solid Support: As used herein, a “solid support” is any material that maintains its shape under conditions useful for practicing methods of the invention, and that can be separated from a liquid phase. Supports that maintain their shape need not be rigid. Indeed, it is contemplated that flexible polymers such as carbohydrate chains, may be used as solid supports, so long as they can be separated from a liquid phase. The present invention is not limited by the type of solid support utilized. A variety of solid supports are contemplated to be useful in the present invention including, but not limited to, a bead, planar surface, controlled pore glass (CPG), a wafer, glass, silicon, diamond, graphite, plastic, paramagnetic bead, magnetic bead, latex bead, super paramagnetic bead, plurality of beads, microfluidic chip, a silicon chip, a microscope slide, a microplate well, a silica gel, a polymeric membrane, a particle, a derivatized plastic film, a glass bead, cotton, a plastic bead, an alumina gel, a polysaccharide, polyvinylchloride, polypropylene, polyethylene, nylon, Sepharose, poly(acrylate), polystyrene, poly(acrylamide), polyol, agarose, agar, cellulose, dextran, starch, FICOLL, heparin, glycogen, amylopectin, mannan, inulin, nitrocellulose, diazocellulose or starch, polymeric microparticle, polymeric membrane, polymeric gel, glass slide, styrene, multi-well plate, column, microarray, latex, hydrogel, porous 3D hydrophilic polymer matrix (e.g., HYDROGEL, Packard Instrument Company, Meriden, Conn.), fiber optic bundles and beads (e.g., BEADARRAY (Illumina, San Diego, Calif.), described in U.S. Pat. Publ. 2005/0164177), small particles, membranes, fits, slides, micromachined chips, alkanethiol-gold layers, non-porous surfaces, addressable arrays, and polynucleotide-immobilizing media (e.g., described in U.S. Pat. Publ. 2005/0191660). In some embodiments, the solid support is coated with a binding layer or material (e.g., gold, diamond, or streptavidin).

The invention provides compositions and methods useful, for example, for the selective amplification of nucleic acid molecules in samples.

Compositions and methods are described for the selective amplification of target nucleic acid segments (e.g., nucleic acid segments present in a sample). Methods of the invention may involve a number of steps but will often employ, as well as possibly additional steps, at least some of the following steps: (1) acquisition of a sample, (2) fragmentation of nucleic acid molecules present in the sample, (3) conversion of nucleic acid in the sample to single-stranded form, (4) contacting the sample of (1), (2) and/or (3) with a probe (e.g., a tagged probe) which is capable of hybridizing (e.g., specifically hybridizing) to one or more target nucleic acid segments which are or might be present in the sample, (5) extension of probes bound to target nucleic acid segments to generate at least partially double-stranded nucleic acid molecules, (6) size selection of nucleic acid molecules generated in (5), (7) capture of size selected nucleic acid molecules (normally via a tag), (8) separation of captured nucleic acid molecules from other mixture components (e.g., nucleic acid molecules which do not contain a tag), and (9) sequencing and/or cloning of captured nucleic acid molecules.

As indicated above, one application for which methods of the invention may be used is to amplify target nucleic acid segments for additional applications (e.g., sequencing). Sequencing and other applications are discussed in more detail elsewhere herein.

Samples may be derived from any number of sources. Since methods described herein may be used to identify and/or amplify target nucleic acid segments, most samples will either be known or suspected to contain one or more target nucleic acid segment.

Samples may be obtained from prokaryotic sources such as bacteria or from eukaryotic sources such as plants, yeast, molds, invertebrates, insects, vertebrates, fish, mammals, rodents, primates and humans. Samples may be acquired from the organism using any of the methods commonly known in the art. In the case of higher eukaryotic organisms the nucleic acid may be isolated from particular organs or tissues such as blood, bone marrow, liver, spleen, kidney, or brain.

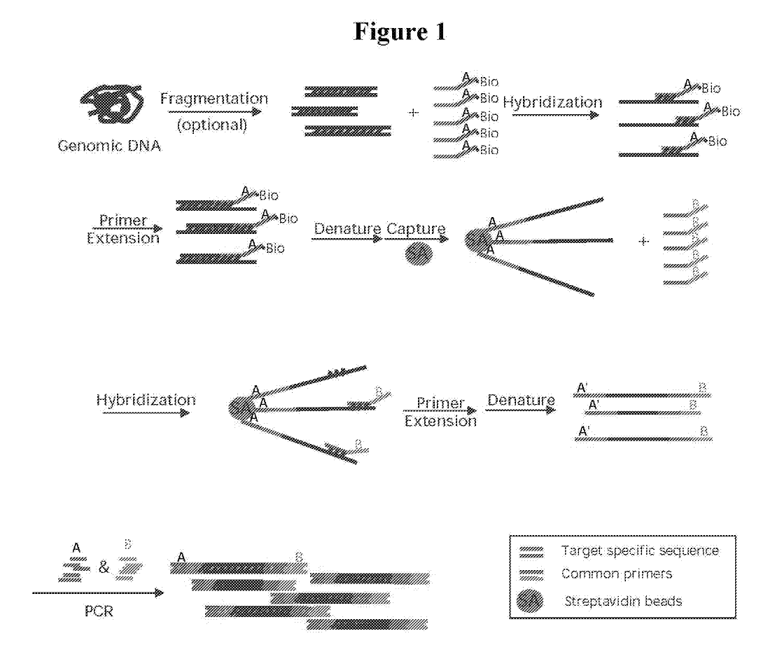

Sample sources include culture media, either cell free or cell containing, as well as cell storage media. In some instances, such samples will be culture media. Culture media may be, for example, designed for the cultivation of prokaryotic or eukaryotic cells. In instances, were target nucleic acid segments are present outside of cells (e.g., target nucleic acid segments of lytic viruses or genomic DNA of cells which have lysed), culture media samples may be subjected to low speed centrifugation (e.g., to remove debris) prior to use in methods of the invention (e.g., methods illustrated in FIG. 1).

Target nucleic acid segments may be (1) RNA or DNA, (2) single or double-stranded, and/or (3) genomic or non-genomic nucleic acids. RNA may also be isolated from an organism and used for selective amplification directly using the methods described herein. Or the isolated RNA may be used to prepare cDNA using methods known in the art. Such cDNA samples may then be used for selective amplification using the methods described herein.

Target nucleic acid segments can be or contain a region of a genome which encodes proteins involved in metabolic, regulatory, or signaling pathways. For example, kinases, growth factors, histocompatibility markers, immunoglobulins, and membrane transport proteins. Nucleic acid sequences encoding proteins associated with particular disease states such as cancer, cardiovascular disease, obesity, and diabetes may also be targets of the selective amplication process. These may include proteins involved in lipid metabolism, fat metabolism, glucose metabolism, blood pressure regulation, toxic compound metabolism among others. Because many of the potential applications for selective amplification may involve analysis of complex biological pathways or systems, in many embodiments multiple target sequences related to multiple proteins involved in a particular pathway or system may be amplified. Target nucleic acid sequences need not be limited to sequences encoding protiens but may include non-coding regions of genes and sequences involved in regulation of gene expression such as promoter sequences. Target nuclei acid sequences can be located at one or more contiguous regions or multiple non-contiguous individual loci.

FIG. 1 shows a diagram of one embodiment of a method of the invention for selective amplification of target nucleic acid sequences. In some embodiments, the nucleic acid may be fragmented. Methods of fragmentation may be mechanical such as shearing or sonication or may be enzyme based such as through the use of restriction enzymes or nucleases. Nucleic acid segments, fragmented and unfragmented) used in the practice of the invention may be of essentially any length (e.g., 100 bp to 20,000 bp in length if fragmented).

For embodiments similar to the one depicted in FIG. 1, a first probe containing two nucleic acid segments may be used to hybridize to the target nucleic acid sequence and serve as primer for subsequent amplification reactions. These two nucleic acid segments may be comprised of a target nucleic acid specific sequence and a common primer sequence. The target nucleic acid specific sequence may specifically hybridize with nucleic acid sequences in or flanking the target sequence. The common primer sequence may be introduced to provide a method of amplifying the products of the selective amplification method. The common primer sequences may also be used to introduce sequences used in downstream applications. As an example, the common primer sequences may be used in next generation sequencing applications such as the P1 and P2 sequences used in the SOLiD system, or adaptor sequences used in Genome Sequencer (Roche/454) or Genetic Analyzer (Illumina).

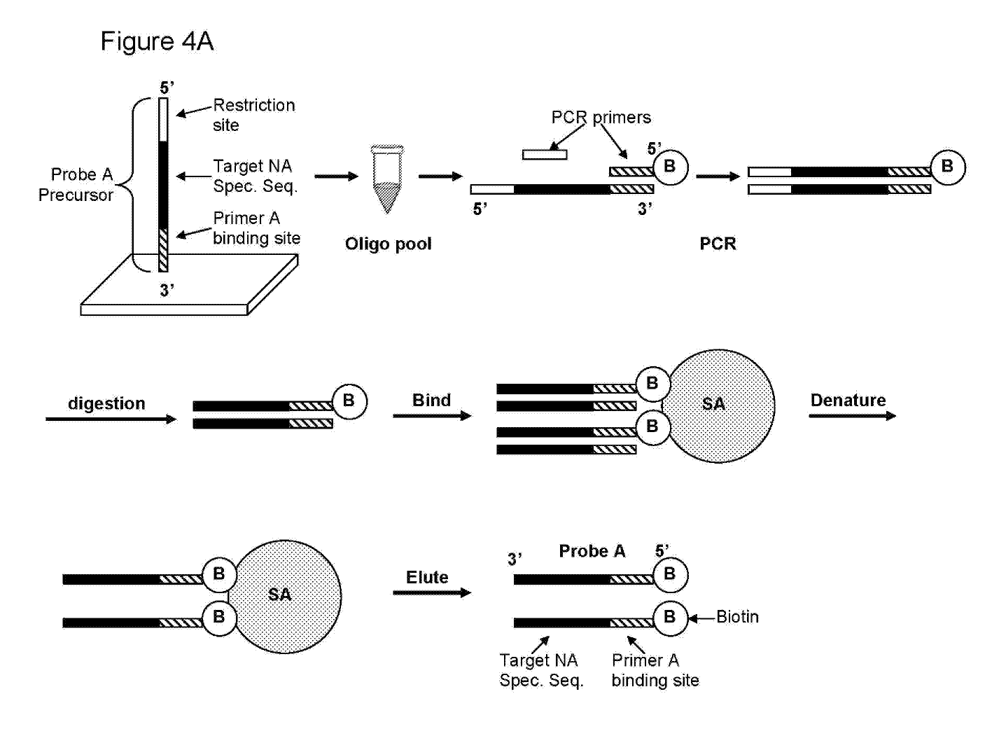

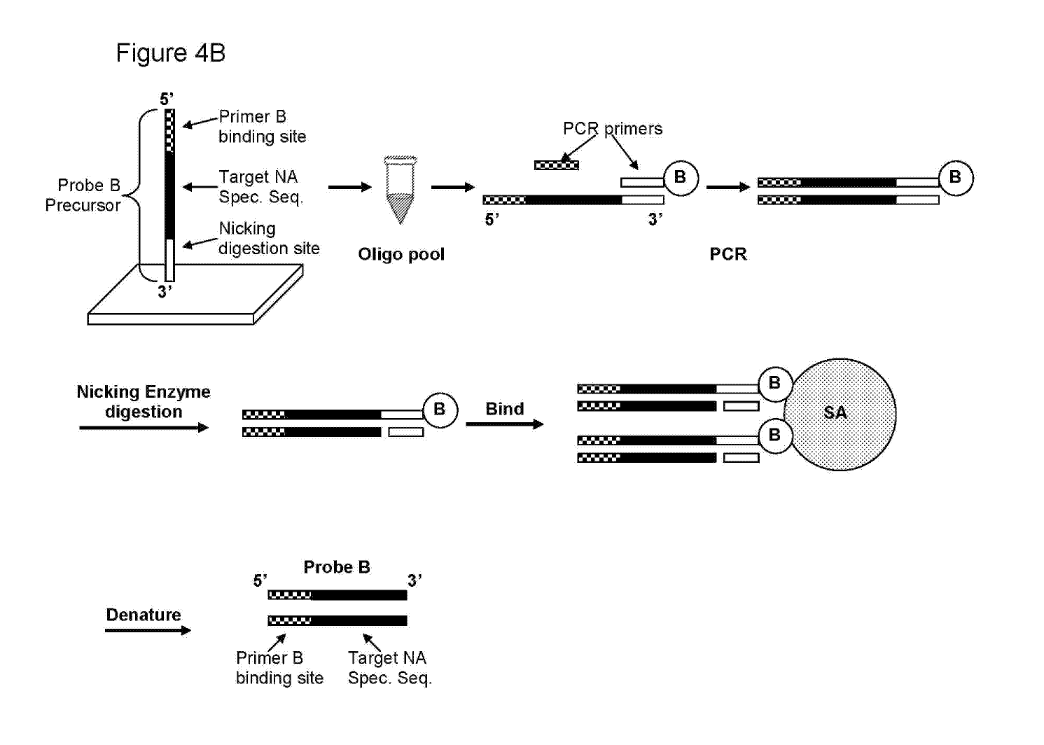

To a large extent, enrichment of desired target nucleic acid segments can be facilitated by the use of probes which contain regions such as those illustrated in FIG. 4. In many instances, one probe will contain a primer binding site, a target nucleic acid segment specific sequence and a tag. In many instances, a second probe will contain a primer binding site, a target nucleic acid segment specific sequence and optionally a tag. Thus, in many instances, selection of nucleic acid segments which are enriched in methods of the invention may be mediated by the use of probes which contain target nucleic acid segment specific sequences.

For purposes of illustration, human cells contain numerous kinases. If a project were to entail comparing the genomic DNA sequences of ten specific kinases in cells obtained from a patient population, cell containing samples from each patient could be processed according to methods of the invention using mixtures of probes designed to enrich genomic DNA encoding each of the ten kinases. In instances where the full length genomic nucleic acid of the target nucleic acid segment could be effectively amplified in a single PCR run (e.g., is less than about 5 kb in length), only twenty probes would be required (e.g., probes designed to hybridize to 5′ and 3′ regions of the segments to be amplified). In instances where the length of the genomic segments are too long to be effectively amplified in a single PCR run (e.g., greater than about 5 kb in length), then multiple primer sets may be used to amplify each target nucleic acid segment. Again for purposes of illustration, assume that each of the kinase genes are 7 kb in length, then two sets of overlapping primers may be used to generate nucleic acid segments which an overlapping region. Since the goal was to compare nucleotide sequences of kinase genes from multiple human samples, the overlapping regions can be used to assemble the complete nucleotide sequences of the genomic DNA. Overlapping oligonucleotides may also be useful to improve sequencing accuracy because these regions have been sequenced from both strands. Nucleic acid segments with overlapping regions may also be assembled through vector insertion (see e.g., Zhu et al., BioTechniques 43:354-359 (1997)).

As indicated above, one of the nucleic acid sequences may further comprise a ligand (e.g., a tag) which is part of an affinity pair. As used herein, “affinity pair” refers to a pair of molecules (for example complementary nucleic acid sequences, protein-ligand, antibody-antigen, protein subunits, nucleic acid binding proteins-binding sites, azide-alkyne “click chemistry”) that can reversibly or irreversibly associate as a result of attractive forces that exist between the molecules. An “affinity pair” includes the combination of a binding molecule or ligand and a corresponding capture element. Examples of affinity pairs may include antigen and specific antibody; antigen and specific antibody fragment; folic acid and folate binding protein; vitamin B12 and intrinsic factor; Protein A and antibody; Protein G and antibody; polynucleotide and complementary polynucleotide; peptide nucleic acid and complementary polynucleotide; hormone and hormone receptor; polynucleotide and polynucleotide binding protein; hapten and anti-hapten; lectin and specific carbohydrate; enzyme and cofactor; enzyme and substrate; enzyme and inhibitor; azide and alkyne; biotin and avidin or streptavidin; and hybrids thereof, and others as known in the art. The ligand may be bound to the 5′ terminus of nucleic acid.

One tag addition method which may be used in the practice of the invention is the CLICK-IT® system. CLICK-IT® chemistry typically involves a copper-catalyzed reaction between an alkyne group and an azide group, to form a covalent bond. Depending, of course, on the molecules employed, this can be used in the generation of small bioorthogonallabeling and detection moieties that react very efficiently and specifically with one another. One example of a nucleoside analog which may be used is EdU (5-ethynyl-2′-deoxyuridine). EdU (available from Life Technologies Corp., Carlsbad, Calif., cat. No. A10044) is readily incorporated into nucleic acids and may be used either directly as a tag or to attach tags to nucleic acids which contain it. The EdU contains the alkyne. Any azide containing molecule, such as 11-Azido-3,6,9-trioxaundecan-1-amine and many fluorescent azides, can react with it (“click” reaction) Aspects of the CLICK-IT® system are described in U.S. Pat. Nos. 7,070,941 and 7,375,234 and U.S. Patent Publication 2007/0190597, the entire disclosures of which are incorporated herein by reference.

While FIG. 4A shows a tag located at the 5′ end of a probe, tags may be located essentially anywhere in the probe. Again using EdU as an example, this nucleoside analog has a relatively small alkyne group and may be incorporated anywhere that it is suitable for incorporation in a nucleic acid. Typically, tags will be incorporated in a probe at locations where they are suitable for directly or indirectly serving their function.

In some embodiments, a bar code sequence (i.e., a signal sequence, which may be a unique nucleotide sequence may be used to identify a particular nucleic acid molecule or group of nucleic acid molecules, such as nucleic acid molecules derived from the sample) may be a part of primer sequences so that different samples may be pooled together for the downstream process for ease of handling and high throughput. In many instances, bar codes will be between 4 and 36 nucleotides (e.g., 4-9, 5-10, 5-15, 5-20, 5-25, 7-15, 7-19, 7-25, etc.) in length. Aspects of bar code systems are described in U.S. Pat. Nos. 6,172,218 and 7,285,384 and U.S. Patent Publications 2009/0068665 and 2008/0269068, the entire disclosures of which are incorporated herein by reference. Bar codes may be used, for example, for multiplex processing of samples.

The invention further provides for methods involving the processing of multiple samples simultaneously. As an example, using the work flow set out in FIG. 1 for purposes of illustration, genomic DNA may be obtained from two or more sources. The DNA present in each of these samples may be fragmented, followed by hybridization to probe A. Further, probe A in this instance contacted with the DNA in each individual sample may contain a unique nucleotide sequence for purposes of identification (e.g., a bar code). While this bar code may be in various places in probe A, it often will be located between the primer binding site and the target nucleic acid specific sequence. Thus, the invention further includes probes contain bar codes and probe sets (including kits containing such sets) which contain bar codes.

Bar code characteristics, in addition to length can vary greatly. Variable parameters include AT/GC ratio and “relatedness” of the bar code sequence to sequence of nucleic acid molecules (e.g., target nucleic acid molecule and/or non-target nucleic acid molecules) potentially present in samples being processed. With respect to AT/GC ratio, considerations for selection of this parameter include desired melting properties of probe-target nucleic acid hybrids, the AT/GC content of nucleic acid molecules in the sample, and the desired AT/GC ratio of other bar code probes. With respect to the last parameter, it may be desirable to employ bar codes which differ in nucleotide sequence but which have similar (e.g., vary by less than 2%, 4%, 6%, 8%, 10%, or 15%) AT/GC ratios. Exemplary AT/GC ratios include 1:1, 1.2:1, 1.3:1, 1.4:1, 1.5:1, 1.6:1, 1:1.1, 1:1.2, 1:1.3, 1:1.4, 1:1.5, 1:1.6, etc. Probes used in methods described herein may also be designed to these AT/GC ratios.

Again using the work flow set out in FIG. 1 for purposes of illustration, in some instances, the DNA derived from different sources will be mixed after the probe A primer extension step. As an example, when amplified target nucleic acid molecules are subject to nucleotide sequencing, bar codes may be used to “de-convolute” sequencing data. In other words, when sequence data is obtained from individual molecules, bar codes sequences may be used to identify the original sample from which the particular molecule sequenced originated from. The invention thus includes, in addition to multiplex processing methods, multiplex sequencing methods.

The common primer portion of the nucleic acid sequence used for hybridization may be at the 5′ end of the molecule. Common primer sequences may be 10-40, 15-35, 15-30 or 15-25 nucleotides in length. Common primer sequences may also be selected so as to not hybridize with sequences with target nucleic acids and/or other nucleic acids likely to be present in the particular samples being processed.

The target specific sequence portion of the probe used for hybridization may be at the 3′ end of the molecule. Target specific sequences may be chosen to hybridize with nucleic acid sequences which flank regions of a genome that are of interest. In some embodiments, the target specific sequences may be 15-100 nucleotides in length. In further embodiments the target specific sequences may be 20-90, 20-80, 20-70, 20-60, 20-50, 25-50, 30-50, 35-50, 20-45, 20-40 or 30-40 nucleotides in length.

Target nucleic acid may be DNA or RNA and may be either single or double-stranded. For embodiments where the nucleic acid is double-stranded the nucleic acid may be denatured before, or as part of, a hybridization protocol.

Various hybridization conditions may be used in the practice of the invention. Depending upon the particular circumstances, conditions of low stringency, moderate stringency or high stringency may be employed. For example, choice of stringency conditions may be determined by factors such as the “complexity” of nucleic acids in the sample (e.g., the number of different sequences present) and the length (as well as AT/GC ratio) of probe regions designed to hybridize to target nucleic acid molecules.

A pair of nucleic acid molecules, such as DNA-DNA, RNA-RNA and DNA-RNA, can hybridize if the nucleotide sequences have some degree of complementarity. As one skilled in the art would understand, single-stranded nucleic acids hydrogen bond to each other, following Watson-Crick base pairing rules. Hybrids can tolerate mismatched base pairs in the double helix, but the stability of the hybrid is influenced by the degree of mismatch. The melting temperature (referred to as the Tₘ) of mismatched hybrids decreases by about 1° C. for every 1-1.5% base pair mismatch. Varying the stringency of the hybridization conditions allows control over the degree of mismatch that will be present in the hybrid. The degree of stringency increases as the hybridization temperature increases and the ionic strength of the hybridization buffer decreases. Stringent hybridization conditions encompass temperatures of about 5-25° C. below the Tₘ of the hybrid and a hybridization buffer having up to 1 M Na⁺. Higher degrees of stringency at lower temperatures can be achieved with the addition of formamide which reduces the Tₘ of the hybrid about 1° C. for each 1% formamide in the buffer solution. Generally, such stringent conditions include temperatures of 20-70° C. and a hybridization buffer containing up to 6×SSC and 0-50% formamide. A higher degree of stringency can be achieved at temperatures of from 40-70° C. with a hybridization buffer having up to 4×SSC and from 0-50% formamide. Highly stringent conditions typically encompass temperatures of 42-70° C. with a hybridization buffer having up to 1×SSC and 0-50% formamide. Different degrees of stringency can be used during hybridization and washing to achieve maximum specific binding to the target sequence. Typically, the washes following hybridization are performed at increasing degrees of stringency to remove non-hybridized polynucleotide probes from hybridized complexes. Thus, the melting point of nucleic acids often relates to the conditions under which two single-stranded nucleic acid molecules will hybridize to each other.

The ionic concentration of the hybridization buffer also affects the stability of the hybrid. Hybridization buffers generally contain blocking agents such as Denhardt's solution, denatured salmon sperm DNA, tRNA, milk powders (BLOTTO), heparin or SDS, and a Na⁺ source, such as SSC (1×SSC: 0.15 M sodium chloride, 15 mM sodium citrate) or SSPE (1×SSPE: 1.8 M NaCl, 10 mM NaH₂PO₄, 1 mM EDTA, pH 7.7). Typically, hybridization buffers contain from between 10 mM-1 M Na⁺. The addition of destabilizing or denaturing agents such as formamide, tetraalkylammonium salts, guanidinium cations or thiocyanate cations to the hybridization solution will alter the Tₘ of a hybrid. Typically, formamide is used at a concentration of up to 50% to allow incubations to be carried out at more convenient and lower temperatures. Formamide can also act to reduce non-specific background when using RNA probes.

As an illustration, a target nucleic acid molecule and a probe can be hybridized to each other at 42° C. overnight in a solution comprising 50% formamide, 5×SSC, 50 mM sodium phosphate (pH 7.6), 5×Denhardt's solution (100×Denhardt's solution: 2% (w/v) Ficoll 400, 2% (w/v) polyvinylpyrrolidone, and 2% (w/v) bovine serum albumin), 10% dextran sulfate, and 20 μg/ml denatured, sheared salmon sperm DNA. One of skill in the art can devise variations of these hybridization conditions. For example, the hybridization mixture can be incubated at a higher temperature, such as about 65° C., in a solution that does not contain formamide.

Another exemplary set of hybridization conditions is as follows: 6×SSC (see stock recipe below), 0.2% SDS, 1×Denhardt's blocking solution, or 1% w/v milk, 10-50 ng/ml denatured probe, with an incubation at 65° C. incubation, with agitation, for 18-24 hours.

Exemplary Recipes: 20×SSC per liter (NaCl 175.3 g (3 molar final in 20×)) and sodium citrate 88.2 g (0.3 molar final in 20×), adjust pH to 7.0 with NaOH); Denhardt's (100×) per liter [Ficoll (Type 400, Pharmacia) 20 g, polyvinylpyrolidone (360) 20 g, bovine serum albumin (e.g., Fraction V, Sigma Chemical Co.) 20 g].

Following hybridization, the nucleic acid molecules can be washed to remove non-hybridized nucleic acid molecule. In some instances such washes will be performed under stringent conditions, or under highly stringent conditions. Typical stringent washing conditions include washing in a solution of 0.5×-2×SSC (e.g., 2×SSC) with 0.1% sodium dodecyl sulfate (SDS) at 55-65° C. (e.g., at 60° C.). Other exemplary wash conditions including 0.5×SSC with 0.1% SDS at 55° C., or 2×SSC with 0.1% SDS at 65° C. One of skill in the art can readily devise equivalent conditions, for example, by substituting SSPE for SSC in the wash solution. Additional washing conditions include washing in a solution of 0.1×-0.2×SSC with 0.1% sodium dodecyl sulfate (SDS) at 50-65° C., 0.1×SSC with 0.1% SDS at 50° C. and 0.2×SSC with 0.1% SDS at 65° C.

After hybridization of the target nucleic acid sequence to a first probe containing the bound ligand (e.g., tag), the first probe may be extended using a polymerase such as such as T4 DNA polymerase and Φ29. In some embodiments the polymerase may be a thermostable polymerase, such as Taq DNA polymerase, Amplitaq Gold (Applied Biosystems). In further embodiments, the thermostable polymerase may be a high fidelity polymerase. Examples of thermostable, high fidelity polymerases include Pfx50 (Invitrogen), Pfu, HotStar HiFidelity Polymerase (Qiagen), Advantage HD Polymerase (Clontech), Phusion™ (Finnzymes Oy), and AccuPrime (Invitrogen).

In some embodiments the extended hybridized molecule may be purified to remove excessive first probes or products of side reactions such as probe dimers or other incomplete reaction products. The purification may be by size exclusion chromatography or other methods know in the art.

After hybridization with the first nucleic acid molecule containing the bound ligand the hybridized and extended molecule may be bound to a solid support. Examples of solid supports include, but not limited to membranes, sepharose beads, magnetic beads, tissue culture plates, glass slides, silica based matrices, membrane based matrices, beads comprising surfaces including but not limited to styrene, latex or silica based materials and other polymers for example cellulose acetate, teflon, polyvinylidene difluoride, nylon, nitrocellulose, polyester, carbonate, polysulphone, metals, zeolites, paper, alumina, glass, polypropyle, polyvinyl chloride, polyvinylidene chloride, polytetrafluorethylene, polyethylene, polyamides, plastic, filter paper, dextran, germanium, silicon, (poly)tetrafluorethylene, gallium arsenide, gallium phosphide, silicon oxide, silicon nitrate and combinations thereof. The capture molecule may be a member of an affinity pair as described above and may bind the ligand that is attached to the hybridized nucleic molecule thereby binding the hybridized and extended nucleic acid to the solid support. Methods of attaching a capture molecule are well known in the art.

In some instances, nucleic acid molecules (e.g., tagged target nucleic acid molecules) may be bound to a solid support. Release of nucleic acid molecules from supports (e.g., solid supports) can occur any number of ways. For example, release could be mediated by enzymatic cleavage of the probe encoded nucleic acid. While essentially any sequence specific cleaving enzyme could be used (e.g., a restriction endonuclease, it will normally be desirable to use a rare cutting enzyme so as to minimize cleavage of desired nucleic acids. A number of rare cutting enzymes are known in the art and can actually be engineered to have desirable properties (Samuelson et al., “Engineering a rare-cutting restriction enzyme: genetic screening and selection of NotI variants” Nucleic Acids Research 34:796-805 (2006)). Typically, rare cutters used in the practice of the invention will recognize nucleotide sequences which are between eight and twenty nucleotides in length (e.g., from about 8 to about 18, from about 10 to about 18, from about 12 to about 18, from about 8 to about 16, from about 8 to about 13, from about 8 to about 11, etc. nucleotides in length). Exemplary rare cutting enzymes which may be used in the practice of the invention include AscI, NotI, PI-PspI, SceI, and SrfI. Another example of a rare cutting enzyme is the HO endonuclease of Saccharomyces cerevisiae (Bakhrat et al., “Homology Modeling and Mutational Analysis of Ho Endonuclease of Yeast” Genetics 166:721-728 (2004)), a LAGLIDADG homing endonuclease that initiates mating-type interconversion. Thus, probes of the invention may contain restriction endonuclease recognition sites. Such sites may be located essentially anywhere within the probe but in many instances will be located in manner which does not alter function of probe sequences. Using probe A as described in FIG. 4 for purposes of illustration, a restriction endonuclease recognition site may be located between the tag and primer binding site. As result, the primer binding site would remain with the target nucleic acid molecule after cleavage. Further, such a restriction endonuclease cleavage site may be used not only for release from a support but also to remove groups associated with target nucleic acid molecule isolation when methods other than those employing supports are used (e.g., dialysis).

Probes of the invention may be synthesized by any of the methods known in the art. In some embodiments probes may be prepared as shown in FIG. 4. FIG. 4 shows schematic representations of methods for preparing probes of the invention and exemplary probes made thereby. While probes may be generated by any suitable method, as set out in FIG. 4A, probe A is made by chemical synthesis on a solid support (e.g., a glass bead). The probe A precursor in FIG. 4 has three regions: (1) a primer binding site (3′ end), (2) a target nucleic acid specific sequence, and (3) a restriction enzyme recognition sequence (5′ end). Once generated, the probe precursor may be amplified by PCR using primers which hybridize to 5′ and 3′ regions of the chemically synthesized nucleic acid molecule. In the work flow shown in FIG. 4, biotin is added to the 5′ end of PCR amplified probe precursors. Restriction endonuclease digestion then result in biotin being attached to the probe at the 5′ terminus with the primer binding site. These biotin containing nucleic acid molecules are then purified through interaction with a solid support to which streptavadin is bound. Elution results in purified single-stranded probe A nucleic acid.

FIG. 4B shows a similar process for generation of probes suitable for use as probe B. In this case, biotin is used to purify the probe B precursor but is not present in the final probe. Further, probe precursor nucleic acid is nicked so that denaturation result in the release of probe B from the solid support.

Non-enzymatic methods for releasing nucleic acid molecule may also be employed. Examples of principles which are the basis for such methods include ion concentration alteration, hydrophobicity/hydrophilicity alterations, and competitive binding/release. With respect to ion concentration alteration, numerous methods are known in the art for the formation of and disruption of molecular interactions through the alterations of ion concentrations. One example is the use of columns which contain carboxymethyl based resins (e.g., sephadex). Molecules which bind to such resins at low ionic strength (e.g., 10 mM Tris-HCl, pH 7.2) can be eluted using higher ionic strength conditions (e.g., 10 mM Tris-HCl, 250 mM NaCl, pH 7.2). The specific condition used for column binding and elution will vary with the molecules employed.

Methods employing hydrophobicity/hydrophilicity alterations for molecular purification and/or isolation are also known in the art and is one of the separation principles of reverse phase HPLC. One common method in HPLC for altering the hydrophobicity/hydrophilicity interaction of a molecule for which purification and/or isolation is sought is through the use of acetonitrile gradients, typically involving starting at a low acetonitrile concentration (e.g., none), followed by increasing the acetonitrile concentration in a linear manner. Of course, whenever a gradient is used in methods described herein, the gradient may be a linear gradient, a logarithmic gradient, or a step gradient.

To facilitate further hybridization reactions, the hybridized and extended molecule may be denatured either before or after binding to the solid support. After the nucleic acid is bound to the solid support unbound material may be removed. The bound nucleic acid may then be hybridized with a second nucleic acid molecule having a common primer sequence at its 5′ end and a target specific sequence at its 3′ end. The second nucleic acid may then be extended using a polymerase. In some embodiments the polymerase will be the same polymerase used for extending the first nucleic acid molecule, e.g. a thermostable high fidelity polymerase.

After removal of polymerase, excessive second probes and other side reaction products the extended second nucleic acid may be released from the solid support by denaturation. The released second molecule may comprise, in order from its' 5′ end, the common second primer sequence, the second target specific sequence, a target nucleic acid sequence, a complementary sequence of the first target specific sequence and a complementary sequence of the first common primer sequence. This product molecule may be further amplified using the common primer sequences using methods known in the art such as polymerase chain reaction.

By complementary, it is meant that two nucleic acid molecules are capable of base-pairing (e.g., hybridize) according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences generally does not interfere with pairing. Whether two nucleic acid molecules will hybridize to each other is determined by factors such as GC/AT content, type of nucleic acid molecules involved (e.g., RNA:DNA, DNA:DNA, RNA:RNA) length of regions of sequence similarity/identity and hybridization conditions. Nucleic acid molecule characteristics and hybridization conditions, as well as other features, which can effect whether two nucleic acid molecules are capable of hybridizing to each other are discussed elsewhere herein.

In further embodiments, the solid support with the bound product of the first polymerization reaction may be reused to produce additional product molecules. The solid support reused in this manner may be used with the same or different probe sequences used in the hybridization step.

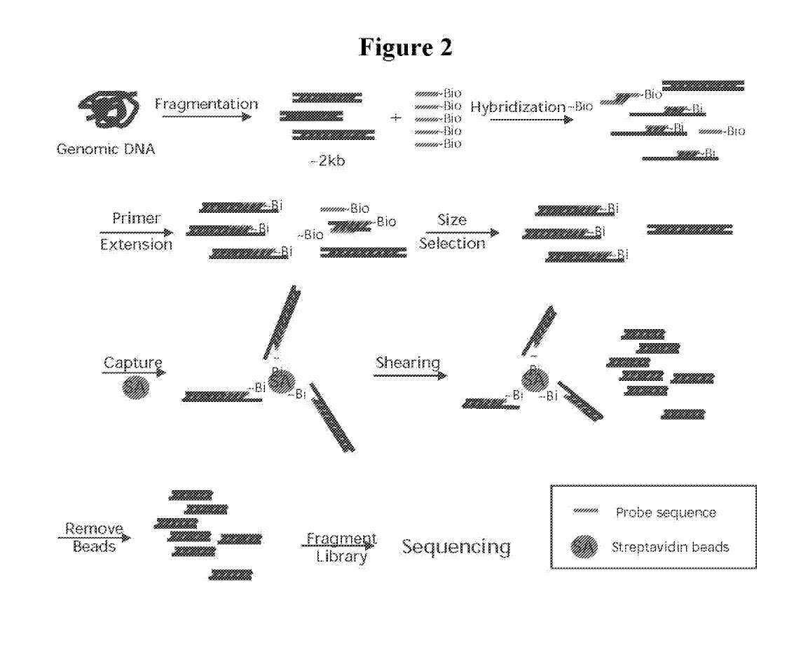

FIG. 2 shows an alternative embodiment of a method for selective replication of target nucleic acid sequences. Methods of fragmentation may be mechanical such as shearing or sonication or may be enzyme based such as the use of restriction enzymes or nucleases. The nucleic acid fragments may be from 100 bp to 20,000 bp, 200 bp to 20,000 bp, 300 bp to 20,000 bp, 500 bp to 20,000 bp, 700 bp to 20,000 bp, 1,000 bp to 20,000 bp, 5,000 bp to 20,000 bp, 10,000 bp to 20,000 bp, 15,000 bp to 20,000 bp, 100 bp to 15,000 bp, 100 bp to 10,000 bp, 100 bp to 5,000 bp, 100 bp to 3,000 bp, 100 bp to 1,000 bp, 100 bp to 500 bp, 100 bp to 300 bp, 200 bp to 5,000 bp, 200 bp to 1,000 bp, 200 bp to 800 bp, 200 bp to 600 bp, 500 bp to 15,000 bp, 500 bp to 10,000 bp, 500 bp to 5,000 bp, 500 bp to 3,000 bp or 500 bp to 2,000 bp in length.

For embodiments similar to the one depicted in FIG. 2, one or more nucleic acid sequences may be used to hybridize to the target nucleic acid sequence. These nucleic acid sequences may comprise a probe sequence. The probe sequence may specifically hybridize with nucleic acid sequences closing to the target sequence. The nucleic acid sequences may further comprise a ligand, which is part of an affinity pair, bound to the 5′ terminus of the molecule.

Probe sequences may be chosen to hybridize with nucleic acid sequences which flank or are in the regions of a genome that is of interest. In some embodiments, the probe sequences may be 15-200 nucleotides in length. In further embodiments the probe sequences may be 15-150, 15-100, 15-90, 15-80, 15-70, 20-50, 25-50, 30-50, 35-50, 20-45, 20-40 or 30-40 nucleotides in length.

In some embodiments a signal sequence, which may be a unique nucleotide sequence 4-36 bp in length used to identify a particular nucleotide molecule, may place between the probe sequence and the ligand.

The target nucleic acid sequence may be DNA or RNA and may be either single or double-stranded. For embodiments where the nucleic acid is double-stranded the nucleic acid may be denatured before, or as part of, a hybridization protocol.

After hybridization of the target nucleic acid sequence to a nucleic acid probe molecule having the bound ligand, the probe sequence may be extended using a polymerase such as T4 DNA polymerase and Φ29 polymerase. In some embodiments the polymerase may be a thermostable polymerase, such as Taq DNA polymerase, Amplitaq Gold (Applied Biosystems). The thermostable polymerase may preferably be high fidelity polymerase. Examples of thermostable high fidelity polymerases include Pfx50 (Invitrogen), Pfu, HotStar HiFidelity Polymerase (Qiagen), Advantage HD Polymerase (Clontech), Phusion™ (Finnzymes Oy), and AccuPrime (Invitrogen). Exemplary extension conditions are set out in the accompanying examples.

In some embodiments, the extended hybridized molecule may be purified to remove excessive nucleic acid probe or products of side reactions such as probe dimers or other incomplete reaction products. The purification may be by size exclusion chromatography or other methods know in the art.

In additional embodiments, extension products may optionally be denatured prior to “capture” of tagged molecules. Denaturation may occur by essentially any suitable means. In many instances, denaturation conditions/methods will be selected which minimize losses in yield of target nucleic acid molecules. One example of the methods which may be used to denature extension products is heating (e.g., placing tubes containing extension products into boiling water). Further, in addition to heating, chemical conditions may be altered to increase the efficiency of denaturation. As an example, solutions which are identical to or approximate those used to generate stringent hybridization conditions may be employed. Providing a local “chemical” environment which facilitates strand separation can be used to allow for denaturation at relatively low temperatures. In some instances, the use of low temperature may be desired. As an example, if a tag is present which may be damaged by temperatures generated in boiling water baths, then denaturation conditions can be adjusted so as to limit tag degradation. Thus, the invention includes the use of denaturation conditions at temperatures less than 98° C. (e.g., from about 40° C. to about 98° C., from about 40° C. to about 80° C., from about 40° C. to about 70° C., from about 40° C. to about 65° C., from about 40° C. to about 60° C., from about 40° C. to about 55° C., from about 40° C. to about 50° C., from about 35° C. to about 50° C., from about 50° C. to about 60° C., from about 50° C. to about 65° C., etc.). Such denaturation conditions may be employed in any one step where denaturation occurs or in all denaturation steps (e.g., denaturation of target nucleic acid prior to hybridization of probe A in the work flow illustrated in FIG. 1.

After hybridization and extension with the probe nucleic acid molecule and, optionally, denaturation, tagged product nucleic acid molecules may be bound to another compound which allows for their separation from untagged nucleic acid molecules. In many instances, tagged product nucleic acid molecules will be separated from untagged nucleic acid molecules by linkage to a solid support. However, tagged nucleic acid molecules may be separated from untagged nucleic acid molecules by means other than the use of solid supports. An example, 5-methyl dCTP can be used with dATP, dGTP and dTTP in the primer extension reaction to protect extended tagged molecules from enzymatic digestion. Thus we can remove untagged molecules by enzymes, eg. restriction endonucleases. After this enzymatic reaction, a size exclusion based purification step may be used for further purification. Because tagged nucleic acid molecules will have substantially higher molecular weight than digested untagged nucleic acid molecules, it allows for separation by size. One method for conjugation and separation of nucleic acid molecules based upon size is set out in Haynes et al., Bioconjugate Chem. 16, 929-938 (2005). Haynes et al. relates to a bioconjugate method of which alters the size of DNA molecules, followed by separation of these molecules by capillary electrophoresis (CE). In particular, this paper discusses the use of branched poly(N-methoxyethyl glycine)s (poly(NMEG)s, a class of polypeptoids) as novel friction-generating entities (referred to as “drag-tags”) for end-on attachment to DNA molecules. It was found that drag was found to scale linearly with total molecular weight, regardless of branch length. Methods such as this may be used for separation of single-stranded or double-stranded nucleic acid molecules.

Further, if tagged nucleic acid molecules are modified in a manner to alter their size any number of size separation methods may be employed (e.g., gel electrophoresis, HPLC, dialysis, sephadex column chromatography, etc.). Similar methods may also be used if tagged nucleic acid molecules are produced which differ from tagged nucleic acid molecules in molecular characteristic other than size (or just size). Examples of such characteristics include hydrophilicity/hydrophobicity and charge. As one skilled in the art would understand, column chromatography and HPLC separation systems can facilitate molecular separation by any number of means. For example, reverse phase HPLC is commonly used to separate molecules based, at leas in part, upon hydrophibic character. Thus, the invention includes methods for separating tagged nucleic acid molecules (e.g., target nucleic acid molecules) from untagged nucleic acid molecules using methods which do not employ a solid support (e.g., by dialysis, centrifugation (e.g., density gradient centrifugation), etc.).

The invention also includes methods for separating tagged nucleic acid molecules (e.g., target nucleic acid molecules) from untagged nucleic acid molecules using methods which do employ a solid support (e.g., by HPLC, gel electrophoresis, sephadex column chromatography, beads, etc.).

The capture molecule (e.g., tag) may be a member of an affinity pair as described above and may bind the ligand that is attached to the hybridized nucleic molecule thereby binding the hybridized and extended nucleic acid to the solid support. Methods of attaching a capture molecule to nucleic acid molecules (as well as other molecules) and solid supports are known in the art and include method set out herein (e.g., via the CLICK-IT® system).

After the nucleic acid is bound to a solid support, unbound material may be removed. The nucleic acid (e.g., double-stranded nucleic acid) may then be released from the solid support. Release of double-stranded nucleic acid may be accomplished by subjecting the bound nucleic acid to shear forces. The shear forces cause the double-stranded nucleic acid to be released from the solid support and may also serve to size the nucleic acid to a relatively uniform size. The stronger the shear forces the shorter the nucleic acid will be. In some embodiments the size of the product nucleic acid may be 50 bp to 100 bp, 50 bp to 150 bp, 50 bp to 200 bp, 100 bp to 200 bp, 100 bp to 300 bp, 100 bp to 400 bp, 200 bp to 300 bp, 200 bp to 400 bp, 200 bp to 500 bp, 200 bp to 600 bp, 200 bp to 700 bp, 200 bp to 800 bp, 200 bp to 900 bp, 200 bp to 1000 bp, 50 bp to 1,000 bp, 50 bp to 900 bp, 50 bp to 800 bp, 50 bp to 700 bp, 50 bp to 600 bp, 50 bp to 500 bp, 50 bp to 400 bp, 50 bp to 300 bp, 50 bp to 1,000 bp, 50 bp to 1,500 bp, 50 bp to 2,000 bp, 50 bp to 2,500 bp, 50 bp to 3,000 bp, 800 bp to 1,500 bp, 800 bp to 2,500 bp, etc. in length.

In some embodiments the immobilized one or more extended probes may be denatured to release the captured single-stranded target nucleic acid from the solid support and the released nucleic acid sequences may be identified. Released target nucleic acid may be used directly or fragmented further before use.

In many instances, target nucleic acid molecules will have undergone primer extensions based upon a work flow identical or similar to that set out in FIG. 1. In such instances, target nucleic acid molecules will contain desired nucleic acid segments flanked by common sequences (e.g., primer binding sites of probes A and B set out in FIG. 4). Thus, once selective enrichment has occurred, target nucleic acid molecules may be amplified (e.g., by polymerase chain reaction, also referred to as PCR).

PCR may also be performed on target nucleic acid molecules which have undergone primer extension using from only one end. The work flow shown in FIG. 1 is designed to generate nucleic acid molecules (e.g., target nucleic acid molecules) which contain common primer binding sites on or near each end. Probe A is used in this work flow to separate certain nucleic acid molecules (e.g., nucleic acid molecules which contain a particular nucleotide sequence) present in samples from other nucleic acid molecules. Thus, the invention includes methods which do not include, for example, primer extension using probe B. Such methods may include, for example, hybridization of probe A to target nucleic acid molecules, followed by separation of these molecules from other nucleic acid molecules in a sample. As may be noted in FIG. 1, probe B binds to a common sequence in target nucleic acid molecules. Such common sequences may be used as primer binding sites for, for example, PCR. Further, primers bound to these primer binding sites may be of a single species or may be contain multiple species designed to hybridize to different target nucleic acid molecules in the sample. Mixed species primers may be useful when target nucleic acid molecules are heterogeneous with respect to nucleotide sequence.

Further, instead of adding terminal nucleotide sequences to target nucleic acid molecules by primer extension, such sequences may be added via ligation. As an example, primer extension using probe B could be replaced with adapter ligation. Methods for ligation adapters to the termini of nucleic acid molecules are known in the art and are set out in U.S. Pat. No. 6,107,023. Thus, the invention includes work flow similar to those set out in FIG. 1 but which are abbreviated in comparison.

The invention also includes kits (e.g., kits for practicing methods of the invention). Thus, in another aspect, the invention provides kits which may be used in conjunction with the invention. Kits according to this aspect of the invention may comprise one or more containers, which may contain one or more components selected from the group consisting of (1) one or more nucleic acid molecules or vectors of the invention, (2) one or more primers, (3) one or more supports (e.g., one or more solid supports), (4) one or more polymerases, (5) one or more reverse transcriptases, (6) one or more buffers, (7) one or more tags or one or more molecules which may be used to prepare tags, (8) one or more restriction endonucleases, (9) one or more vectors, (10) one or more sets of instructions for using kit components and/or practice methods described here, and the like.

One exemplary kit of the invention could include (1) collections/sets of molecules suitable use as probes A and probes B (e.g., probes with sequence specific regions for the amplification of a subset of human kinase genes), where the A probes contain a biotin tag, (2) streptavidin modified magnetic beads, (3) Pfx50 DNA polymerase, and (4) instructions for using kit components for the selective enrichment of nucleic acid molecules designed to hybridize to kit probes.

Kits of the invention can also be supplied with primers. These primers will often be designed to anneal to molecules having specific nucleotide sequences (e.g., primer binding sites of probe A and/or probe B sequences). For example, these primers can be designed for use in PCR to amplify a particular nucleic acid molecule. Further, primers supplied with kits of the invention can be sequencing primers designed to hybridize to vector sequences. Thus, such primers will generally be supplied as part of a kit for sequencing nucleic acid molecules which have been inserted into a vector.

One or more buffers (e.g., one, two, three, four, five, eight, ten, fifteen) may be supplied in kits of the invention. These buffers may be supplied at working concentrations or may be supplied in concentrated form and then diluted to the working concentrations. These buffers will often contain salt, metal ions, co-factors, metal ion chelating agents, etc. for the enhancement of activities of the stabilization of either the buffer itself or molecules in the buffer. Further, these buffers may be supplied in dried or aqueous forms. When buffers are supplied in a dried form, they will generally be dissolved in water prior to use. Examples of buffers suitable for use in kits of the invention are set out in the following examples.

Supports suitable for use with the invention (e.g., solid supports, semi-solid supports, beads, multi-well tubes, etc., described above in more detail) may also be supplied with kits of the invention.

Kits of the invention may contain virtually any combination of the components set out above or described elsewhere herein. As one skilled in the art would recognize, the components supplied with kits of the invention will vary with the intended use for the kits. Thus, kits may be designed to perform various functions set out in this application and the components of such kits will vary accordingly.

### EXAMPLES

### Example 1

### Selective Amplification of Kinase Related Amplicons

Protein phosphorylation is an important biochemical pathway in biological systems. Selective amplification of kinase related genes was selected as a model system for testing the effectiveness of the amplification methods described herein. The targets chosen for amplification were randomly chosen from those published in PNAS, 105(27):9296-9301, 1998.

Five micrograms of a male human genomic DNA (Promega, Madison, Wis., cat. No. G1471) was sheared to 5-10 kb using a HydroShear (Genomic Solutions, Ann Arbor, Mich.) standard assembly at speed code 18. In a PCR tube, 500 ng sheared DNA was mixed with 1 μl of first oligonucleotide mix A (also referred to herein as a probe mix), targeting 44 kinase genes at 1 μM each, in Pfx50 PCR buffer (Invitrogen, Carlsbad, Calif., part no. 56061). The DNA solution (total 19 μl) was denatured at 94° C. for 2 minutes and annealed at 58° C. for 60 minutes. One microliter of a mixture of dNTP at 5 mM each and 2.5 units of Pfx50 DNA polymerase (Invitrogen, Carlsbad, Calif., cat. no. 12355-012) was added into the PCR tube at the end of the annealing reaction. The tube was further incubated at 58° C. for 10 minutes, then 68° C. for 15 minutes. The first primer extended products were purified with a PureLink PCR Micro column (Invitrogen, Carlsbad, Calif., cat. no. K3100-50) and eluted in 20 μl of elution buffer to remove excess oligonucleotide mix A.

The purified extended products were bound to DynaBead M-270 Streptavidin (Invitrogen, Carlsbad, Calif., catalog no. 653.05) in 1× bead binding buffer. To facilitate the bead capture of the biotinylated DNA strand, the DNA and bead mixture were heated at 90° C. for 1 minute and chilled on ice for 1 minute before incubation on a rotator for 15 minutes at room temperature. The beads with captured DNA were washed with 0.1 N NaOH twice and 1×TE pH. 8.0 three times.

The washed beads were added to 19 μl of 1×Pfx50 PCR buffer which contained 1 μl of the second oligonucleotide mix B (also referred to as probe mix B) at 1 μM each. The beads mixture was incubated at 94° C. for 30 seconds and 58° C. for 15 minutes before addition of 1 μl of mixture of dNTP at 5 mM each and 2.5 U Pfx50 DNA polymerase. The beads mixture was further incubated at 58° C. for 10 minutes, then 68° C. for 10 minutes. After the incubation, 20 μl of 2× bead binding buffer was added. The bead mixture was incubated at 25° C. for 15 minutes and washed with TE buffer three times before denaturing with 40 μl of 0.1N NaOH. The supernatant which contained single-stranded second extended targets was collected. After addition of 5 μl of 3M Sodium Acetate pH 5.3, the collected supernatant was purified using a PureLink PCR Micro column and eluted in 10 μl elution buffer. PCR reactions were set up using the common first and second primers in Platinum SuperMix High Fidelity (Invitrogen, cat. no. 12532-016) and amplified for 30 cycles.

SYBR green based qPCR (Invitrogen, Carlsbad, Calif., cat. no. 11760-500 manual) was performed using each pair of target specific primers to determine enrichment efficiency. Of 44 targets, 43 were enriched with 88% of the targets enriched within a ten fold difference and 100% of the targets enriched within a 64 fold difference.

Sanger sequencing was performed on 20 clones derived from products of the amplification reactions. There was one sequence failure due to mixed clone types. There were two cloning failures due to a missing insert. The remaining 17 clones provided usable sequence information. Of these 17 sequences, 12 were identified from the target sequences; four were identified as the result of primer mismatch and one as an unmatched clone.

### Example 2

### Selective Amplification of 8 HLA and 12 Kinase Amplicons

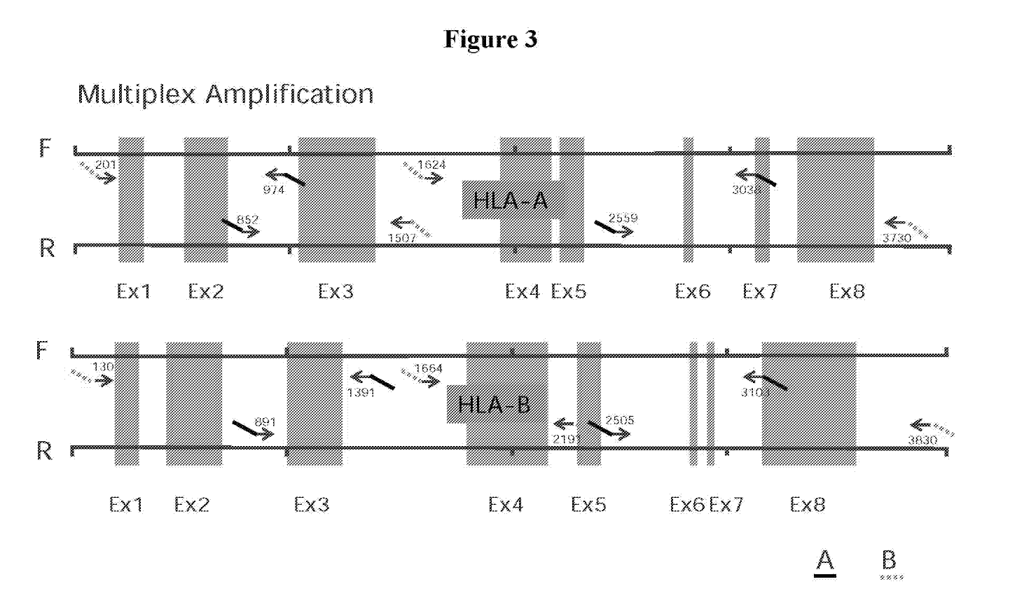

A comparison of selective amplification of 8 HLA related and 12 kinase related amplicons was made. The kinase amplicons (Table 1) chosen were those with a higher Tₘ for the primer in the original 44 kinase targets used in Example 1. The HLA amplicons chosen are shown in FIG. 3.

TABLE 1

| Kinase | Chro- | Left Primer | Right Primer | GC % of | Amplicon |
|---|---|---|---|---|---|
| Target | mosome | Length (nt) | Length (nt) | Amplicon | Size (bp) |
| 291976 | 13 | 22 | 23 | 40.5 | 175 |
| 293531 | 1 | 23 | 26 | 44.6 | 186 |
| 292299 | 7 | 23 | 22 | 47.7 | 373 |
| 292065 | 17 | 21 | 21 | 53.1 | 192 |
| 293523 | 1 | 21 | 26 | 56.5 | 214 |
| 293613 | 20 | 23 | 23 | 58.9 | 224 |
| 293605 | 20 | 22 | 21 | 59.9 | 242 |
| 292182 | 3 | 21 | 22 | 61.6 | 295 |
| 293612 | 20 | 21 | 24 | 62.4 | 322 |
| 292071 | 17 | 20 | 23 | 63.7 | 298 |
| 292243 | 5 | 21 | 21 | 65.9 | 194 |
| 293609 | 20 | 21 | 22 | 67.9 | 290 |

In a PCR tube, 500 ng of a male human genomic DNA (Promega), unsheared, was mixed with 1 μl of first oligonucleotide mix A (targeting 8 HLA amplicons and 12 kinase amplicons at 1 μM each) in Pfx50 PCR buffer (Invitrogen). In another two PCR tubes, the same amount of the same genomic DNA was mixed with 1 μl of first oligonucleotide mix A targeting 8 HLA amplicons or 12 kinase amplicons at 1 μM each in Pfx50 PCR buffer, respectively. The DNA solutions (total 19 μl each) were denatured at 94° C. for 2 minutes; annealed at 68° C. for 15 minutes and 65° C. for 15 minutes. 1 μl of mixture of dNTP at 5 mM each and 2.5 U Pfx50 DNA polymerase was added into each PCR tube at the end of annealing reaction. The tubes were further incubated at 65° C. for 5 minutes; 68° C. for 8 minutes; 94° C. for 2 minutes; then chilled on ice. The first primer extended products were purified with PureLink PCR Micro columns and eluted in 20 μl elution buffer to remove excess oligonucleotide mix A (also referred to as probe mix A).

The purified extended products were incubated with 10 μl of DynaBead M-270 Streptavidin in 1× bead binding buffer on a rotator for 30 minutes at room temperature. The beads with captured DNA were washed with 0.1 N NaOH twice and 1×TE three times.

The washed beads were added into 19 μl of 1×Pfx50 PCR buffer which contained 1 μl of the second oligonucleotide mix B (also referred to as probe mix B) at 1 μM each. The beads mixtures from three samples were incubated at 94° C. for 30 seconds; 68° C. for 10 minutes; 63° C. for 10 minutes before addition of 1 μl of mixture of dNTP at 5 mM each and 2.5 U Pfx50 DNA polymerase. The beads mixtures were further incubated at 63° C. for 5 minutes; 68° C. for 8 minutes; chilled on ice. One μl of 0.5M EDTA was added to inactivation DNA polymerase. To recapture all the biotinylated fragments, 20 μl of 2× bead binding buffer was added. The beads mixtures were incubated at 25° C. for 15 minutes and washed with TE for three times before denaturing with 50 μl of 0.1N NaOH twice. The supernatants which contained single-stranded second extended targets were collected. After addition of 10 μl of 3M Sodium Acetate pH 5.3, the collected supernatants were purified with PureLink PCR Micro columns and eluted in 20 μl elution buffer E1. PCR reactions were set up using the common first and second primers in Platinum SuperMix High Fidelity (Invitrogen) and amplified for 25 cycles. After PCR amplification, the PCR products were run through a PCR micro column and eluted in 40 μl of E1.

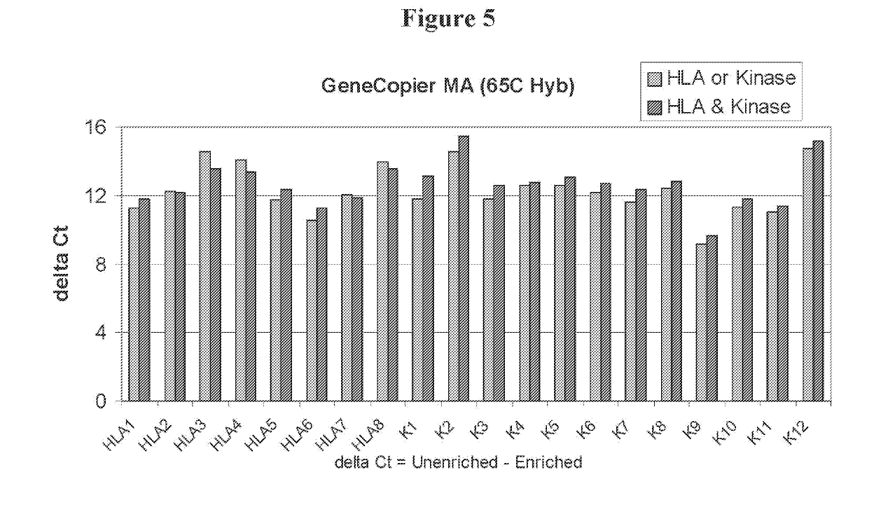

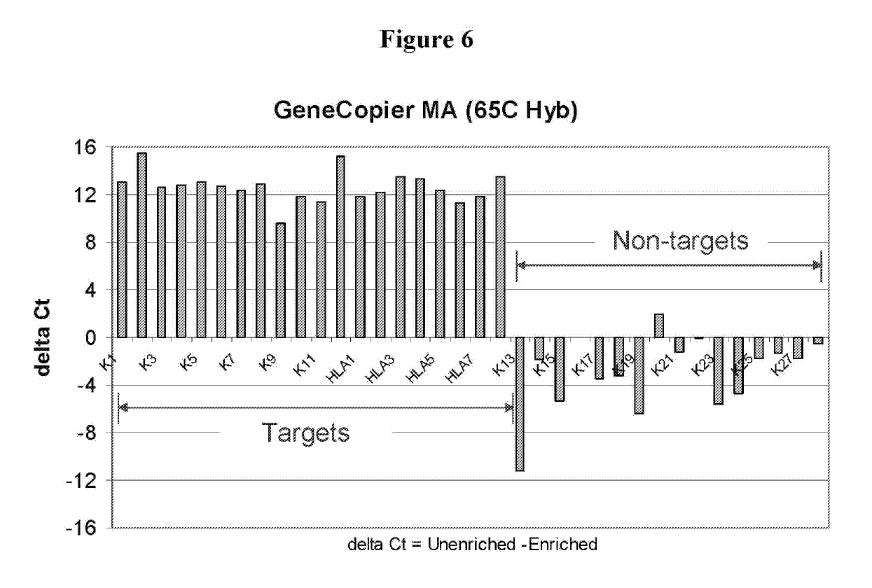

SYBR green based qPCR was performed using each pair of target specific primers to determine enrichment efficiency. The results are shown in FIGS. 5 and 6.

The HLA targets are ranged from 650 bp to 1,450 bp; the kinase targets are ranged from 175 bp to 373 bp. The results showed there was no significant different no matter if these two different size groups of target were selectively amplified together or separately. Compared with the hybridization/annealing condition (58° C. for 60 minutes) used in the Example 1, a higher hybridization temperature (68° C. for 15 minutes and 65° C. for 15 minutes) in this example resulted in more uniform enrichment with greater specificity for the targeted amplicons.

### Example 3

### Improved Selective Amplification Efficiency

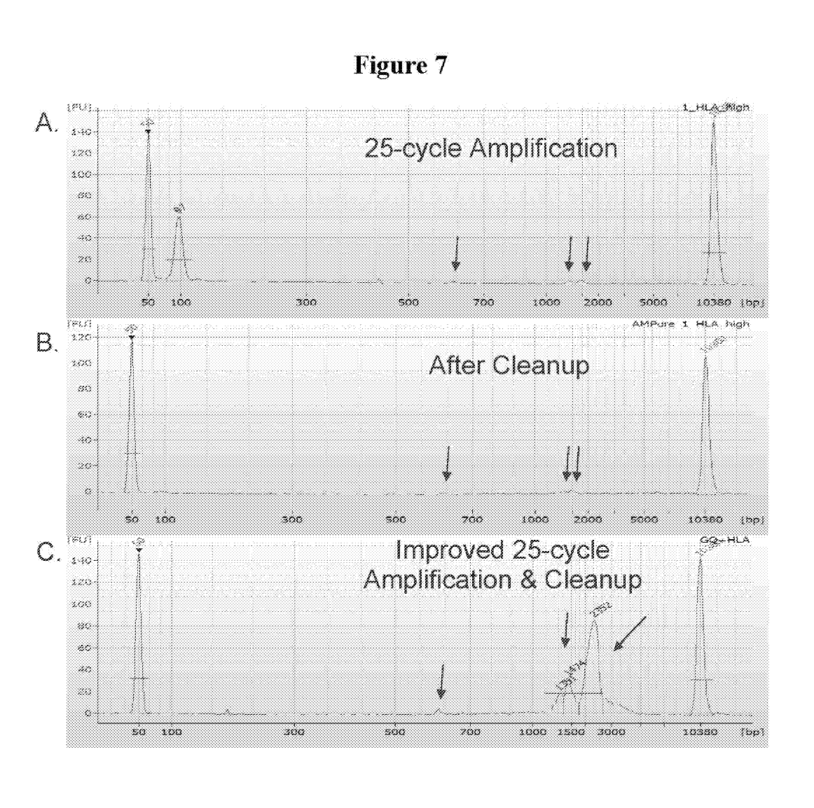

The selective amplification method was further optimized with improving removal of excessive oligonucleotides, longer beads capture and better bead wash. The improved results are shown in FIG. 7C compared with performance before these improvement in FIGS. 7A and 7B.

### Example 4

### Comparison of Target Nucleotide Size and Amplification Efficiency

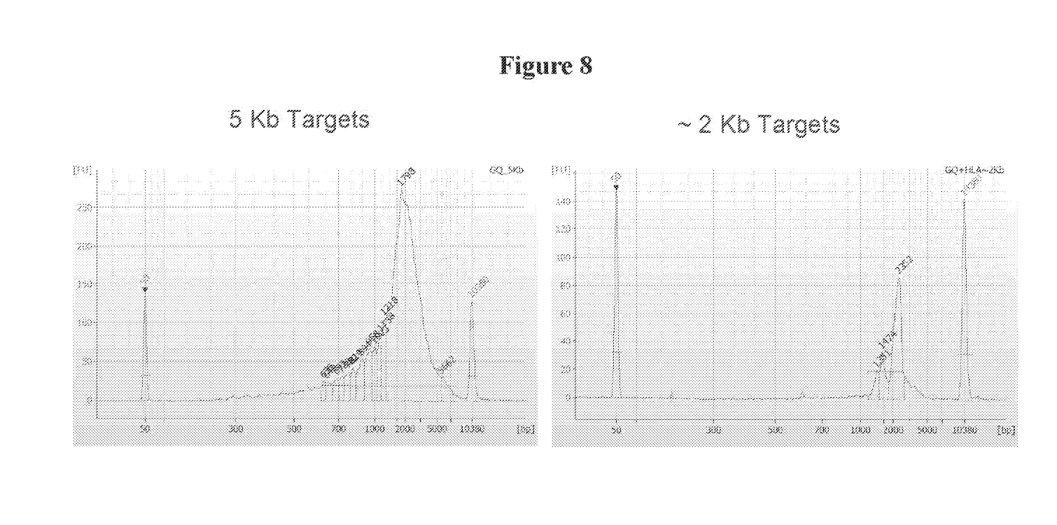

To evaluate the efficiency of amplification on different size of amplicons, selective amplification of approximately 50 target genes involved in various metabolic pathways were performed. The sizes of these targeted amplicons are either 650 bp to 2,400 bp (most around 2,000 bp) or 4,500 bp to 5,500 bp. The Bioanalyzer electropherogram results of this comparison are shown in FIG. 8. The results showed that with the experimental condition used less than 2,500 bp amplicons can be specifically amplified very well, but there are many shorter non-specific amplified products for ˜5,000 bp amplicon targets.

### Example 5

### Comparison of Selective Amplification with Standard Amplification by Polymerase Chain Reaction

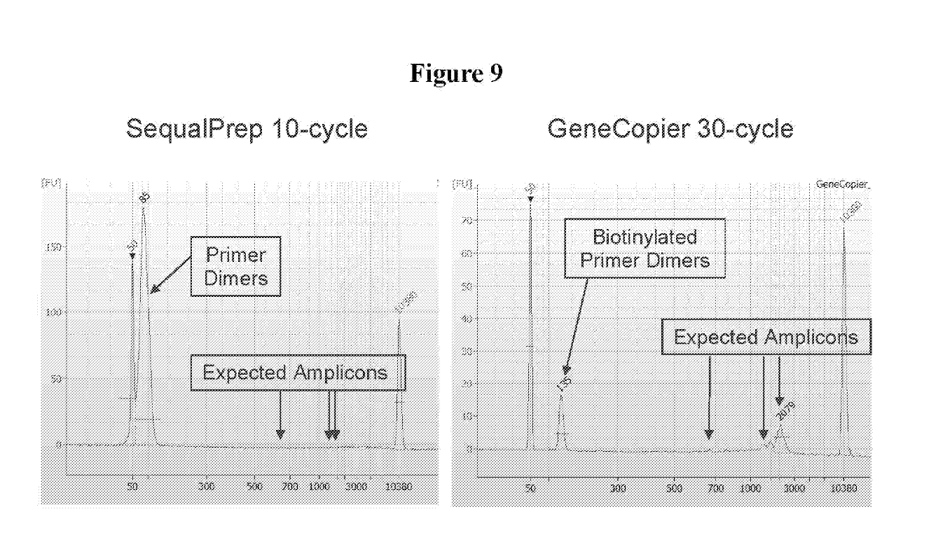

To compare the efficiency of selective amplification to that of standard multiplex PCR 58 amplicons, which sizes ranged from 650 bp to 2,400 bp were amplified by the method described in Example 6 and compared to the amplification of the same sequences using the SequalPrep kit (Invitrogen). The results of this analysis are shown in FIG. 9. The selective amplification method can amplify target amplicons specifically for up to 30-cycle of PCR; the standard multiplex method can not amplify the targets specifically beyond 10-cycle of PCR due to accumulation of overwhelming large amount of PCR side products, such as primer dimmers

### Example 6

### Conditions for Amplification of Selected Amplicons

In a PCR tube, 225 ng of a male human genomic DNA (Promega), unsheared, may be mixed with 1 μl of first oligonucleotide mix A (0.1 to 1 μM each), 0.5 μl of dNTP (10 mM each) and 2.5 U hot start Pfx50 DNA polymerase in Pfx50 PCR buffer (Invitrogen) in a total volume of 20 μl. The DNA solution may be denatured at 94° C. for 2 minutes; annealed at 68° C. for 15 minutes and 65° C. for 15 minutes; denatured again at 94° C. for 2 minutes; kept at 4° C. until further processing. The first primer extended products may be purified with a PureLink PCR Micro column and eluted in 20 μl elution buffer to remove excess oligonucleotide mix A.

The purified extended products may be incubated with 10 μl of DynaBead M-270 Streptavidin in 1× bead binding buffer on a rotator for 60 minutes at room temperature. (Note: the bead incubation time may depend on the size of the primer extended product. The 60 minute reaction is for up to 2 kb targets.) The beads with captured DNA may be washed with 0.1 N NaOH twice and 1×TE three times.

The washed beads were added into 20 μl of second primer extension reaction solution which included 1 μl of the second oligonucleotide mix B (0.1 to 1 μM each) and 0.5 μl of dNTP (10 mM each) and 2.5 U hot start Pfx50 DNA polymerase in 1×Pfx50 PCR buffer (Invitrogen). The PCR tube containing these beads mixture was incubated on a thermocycler as following: 94° C. for 30 seconds; 68° C. for 15 minutes; 65° C. for 10 minutes; kept at 4° C. at last. One μl of 0.5M EDTA was added to inactivation DNA polymerase.

To recapture all the biotinylated fragments, 20 μl of 2× bead binding buffer was added. The beads mixture was incubated at 25° C. for 15 minutes and washed with TE for three times before denaturing with 50 μl of 0.1N NaOH twice. The supernatant which contained single-stranded second extended targets was collected. After addition of 10 μl of 3M Sodium Acetate pH 5.3, the collected supernatant was purified with PureLink PCR Micro column and eluted in 10 μl elution buffer E1. PCR reactions were set up using the common first and second primers in Platinum SuperMix High Fidelity (Invitrogen) and amplified for 25-30 cycles. After PCR amplification, the PCR products were run through a PCR micro column and eluted in 40 μl of E1.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific embodiments, it should be understood that the invention should not be unduly limited to such specific embodiments.

1-55. (canceled)

56. A method for amplification of one or more target nucleic acid molecules comprising:
(a) hybridizing the one or more target nucleic acid molecules with a first probe comprising,
(i) at the 3′ end, a first target nucleic acid specific sequence,
(ii) a first primer sequence at the 5′ end of the first probe, and
(iii) a tag attached to the 5′ end of the first probe or any part of first primer sequence,
(b) extending the first probe,
(c) contacting the extended first probe with a solid support comprising a capture molecule which binds the tag,
(d) hybridizing the extended first probe with a second probe comprising
(i) at the 3′ end, a second target nucleic acid specific sequence, and
(ii) a second primer sequence at a 5′ end of the second probe,
(e) extending the second probe, and
(f) releasing the extended second probe.

57. The method of claim 56, wherein the released second probe is amplified using the first primer sequence and the second primer sequence.

58. The method of claim 56, wherein the extended first probe is denatured prior to contacting the solid support.

59. The method of claim 56, wherein the one or more target nucleic acid molecules is DNA.

60. The method of claim 56, wherein the one or more target nucleic acid molecules is RNA.

61. The method of claim 56, wherein the one or more target nucleic acid molecule is fragmented.

62. The method of claim 56, wherein the first probe is extended by use of a reverse transcriptase when one or more target nucleic acid molecule is RNA.

63. The method of claim 56, wherein the capture molecule is one or more molecules of one member of an affinity pair.

64. The method of claim 63, wherein the affinity pair is selected from the group consisting of antigen and specific antibody; antigen and specific antibody fragment; folic acid and folate binding protein; vitamin B12 and intrinsic factor; Protein A and antibody; Protein G and antibody; polynucleotide and complementary polynucleotide; peptide nucleic acid and complementary polynucleotide; hormone and hormone receptor; polynucleotide and polynucleotide binding protein; hapten and anti-hapten; lectin and specific carbohydrate; enzyme and cofactor; enzyme and substrate; enzyme and inhibitor; azide and alkyne; biotin and avidin or streptavidin.

65. The method of claim 56, wherein the first probe further comprises a barcode sequence.

66. The method of claim 56, wherein the second probe further comprises a barcode sequence.

67. The method of claim 56, wherein the first and second probe each further comprise a barcode sequence.

68. The method of claim 56, wherein steps (d)-(f) are repeated.

69. The method of claim 56, wherein the solid support is selected from the group consisting of a bead, a magnetic bead, a column, a filter, a plate and a slide.

70. A method for amplification of one or more target nucleic acid molecules comprising:
(a) hybridizing the one or more target nucleic acid molecules with one or more probes, wherein the one or more probes comprises:
(i) a target nucleic acid specific sequence;
(ii) a tag attached to a 5′ end of the one or more probes, and/or
(iii) a signal sequence between the target specific polynucleotide sequence and the tag attached to the 5′ end of the one or more probes,
(b) extending the one or more probes enzymatically, and
(c) contacting the one or more extended probes with a solid support comprising a capture molecule which binds the tag.

71. The method of claim 70, wherein the one or more target nucleic acid molecules is DNA.

72. The method of claim 70, wherein the one or more target nucleic acid molecules is RNA.

73. The method of claim 70, wherein the one or more target nucleic acid molecules is fragmented.

74. The method of claim 70, wherein the one or more probes are extended by use of a reverse transcriptase when the one or more target nucleic acid molecules are RNA.

75. The method of claim 70, wherein the capture molecule is one or more molecules of one member of an affinity pair.

76. The method of claim 75, wherein the affinity pair is selected from the group consisting of antigen and specific antibody; antigen and specific antibody fragment; folic acid and folate binding protein; vitamin B12 and intrinsic factor; Protein A and antibody; Protein G and antibody; polynucleotide and complementary polynucleotide; peptide nucleic acid and complementary polynucleotide; hormone and hormone receptor; polynucleotide and polynucleotide binding protein; hapten and anti-hapten; lectin and specific carbohydrate; enzyme and cofactor; enzyme and substrate; enzyme and inhibitor; azide and alkyne; biotin and avidin or streptavidin.

77. The method of claim 70, wherein the solid support is selected from the group consisting of a bead, a magnetic bead, a column, a filter, a plate and a slide.

78. A method for selectively increasing the abundance of one or more target nucleic acid molecules, the method comprising:
(a) obtaining a sample which contains one or more target nucleic acid molecules and one or more non-target nucleic acid molecules;
(b) incubating the sample under conditions suitable for converting double stranded nucleic acid molecules to single stranded nucleic acid molecules, thereby forming a first reaction mixture;
(c) contacting the first reaction mixture of step (b) with a probe under conditions suitable to allow for the probe to hybridize to the one or more target nucleic acid molecules, wherein the probe comprises at least a sequence complementary to the one or more target nucleic acid molecules, a primer binding site, and a tag, thereby forming a second reaction mixture;
(d) contacting the second reaction mixture of step (c) with a polymerase under conditions suitable for primer extension to form one or more tagged double stranded target nucleic acid molecule, wherein the one or more double stranded target nucleic acid molecule comprise a primer binding site and a tag;
(e) contacting the one or more tagged double stranded target nucleic acid molecules formed in step (d) with a solid support under conditions which allow for binding of the one or more double stranded target nucleic acid molecules to the solid support; and
(f) washing of the solid support formed in step (e) to remove the one or more non-target nucleic acid molecules.

79. The method of claim 78, further comprising the step of removing the tagged double stranded target nucleic acid molecules from the solid support.

80. The method of claim 78, wherein the tagged double stranded target nucleic acid molecules are amplified by polymerase chain reaction after removal from the from the solid support.

81. The method of claim 78, wherein nucleic acid molecules present in the sample are fragmented prior to entry into step (b).

82. The method of claim 78, wherein the tag is either biotin or contains a reactive azide group.

83. The method of claim 78, wherein the first reaction mixture is contacted with a mixture of probes which differ from each other in nucleotide sequence.

84. A collection of two or more probes which differ from each other in nucleotide sequence, in which each probe comprises a sequence complementary to at least part of a naturally occurring nucleic acid molecule, a primer binding site, and a tag.

85. The collection of probes of claim 84, wherein between 2 and 40 probes are present.

86. The collection of probes of claim 84, wherein sequence complementary to at least part of a naturally occurring nucleic acid molecule is between 10 and 100 nucleotides in length.

87. The collection of probes of claim 84, wherein the primer binding site is between 10 and 100 nucleotides in length.

88. The collection of probes of claim 84, wherein the probes which differ from each other in nucleotide sequence are in the same container.

89. The collection of probes of claim 84, wherein the probes which differ from each other in nucleotide sequence are each in different containers.

90. The collection of probes of claim 84, wherein the probes are dissolved in an aqueous solution.

91. The collection of probes of claim 84, wherein the probes contain a bar code sequence.
